(19) United States
(12) Patent Application Publication
(10) Pub. No.: US 2017/0065257 A1
(43) Pub. Date: Mar. 9, 2017

(54) ULTRASOUND DIAGNOSTIC DEVICE AND ULTRASOUND IMAGE PROCESSING METHOD

(71) Applicant: Hitachi, Ltd., Tokyo (JP)

(72) Inventors: Masashi NAKAMURA, Tokyo (JP); Masaru MURASHITA, Tokyo (JP); Hajime SAKASHITA, Tokyo (JP); Eiji KASAHARA, Tokyo (JP); Noriyoshi MATSUSHITA, Tokyo (JP)

(73) Assignee: Hitachi, Ltd., Tokyo (JP)

(21) Appl. No.: 15/121,826

(22) PCT Filed: Oct. 8, 2014
(86) PCT No.: PCT/JP2014/076940

§ 371 Date: Aug. 26, 2016

(30) Foreign Application Priority Data
Feb. 27, 2014 (JP) 2014-037116

(51) Int. Cl. A61B 8/08; A61B 8/00; A61B 8/02

(52) U.S. Cl. CPC A61B 8/5223; A61B 8/02; A61B 8/5207; A61B 8/461; A61B 8/0866

(57) ABSTRACT

In the present invention, a reference frame selection unit selects a reference frame from among a sequence of frames showing the heart of a fetus. A candidate region group setting unit sets a candidate region group for each of the frames that constitute the sequence of frames. A correlation value calculation unit calculates, for each candidate region, a correlation value between the reference frame and all the other frames. Due to this, a plurality of correlation value waveforms corresponding to the plurality of candidate regions is generated. A stabilized waveform portion specification unit specifies a stabilized waveform portion for each correlation value waveform. A stabilized region specification unit specifies, from among the plurality of stabilized waveform portions, the stabilized waveform portion having the highest degree of stabilization (in other words, the candidate region having the highest degree of stabilization). A heart rate calculation unit calculates heartbeat information (heart rate, etc) for the fetus on the basis of the stabilized waveform portion having the highest degree of stabilization.

### TECHNICAL FIELD

The present invention relates to an ultrasound diagnostic device, and in particular, to an ultrasound diagnostic device for acquiring period information from a periodically moving organ.

### BACKGROUND ART

For the heart of a fetus, directly measuring a heart rate and other values using an electrocardiograph or the like is difficult. When an ultrasound diagnostic device is used, however, information such as the heart rate can be acquired.

In an ultrasound diagnostic device disclosed in Patent Document 1, for example, a plurality of tomographic images representing the heart of a fetus are used for performing operation to calculate correlation between a reference tomographic image and all other tomographic images. A heart rate of the fetus is calculated from a correlation value waveform representing the result of the calculation.

On the other hand, in an ultrasound diagnostic device disclosed in Patent Document 2, motions of the body and the heart of a fetus are analyzed based on ultrasound images, to thereby obtain a waveform representing a change of the body and a waveform representing the motion of the heart. A heart rate of the fetus is calculated based on the waveform representing the motion of the heart from which the waveform representing the change of the body is subtracted.

### CITATION LIST

### Patent Literature

Patent Document 1: JP 2013-198635 A
Patent Document 2: JP 2013-198636 A

### SUMMARY OF INVENTION

### Technical Problem

When heartbeat information is calculated from a correlation value waveform, the accuracy of calculating the heartbeat information greatly depends on a way of setting a region of interest which is a target of correlation value calculation. For example, in a case where the region of interest is set in a portion of a heart where periodic motion of the heart is unstable, a stable correlation value waveform is not obtained, which raises a problem of decreased accuracy of measuring heartbeat information. It is desirable to set the region of interest at an appropriate position or in an appropriate size.

In particular, the heart of a fetus is very small and is very likely to move. Further, boundaries of the heart represented on an ultrasound image are unclear in many cases. This makes it difficult for a user to manually specify, with stability, a site of the heart where the heart periodically moves.

An object of the present invention is to improve the accuracy of measuring period information on a periodically moving organ in an ultrasound diagnostic device. Another object of the present invention is to lessen or eliminate a burden shouldered by a user to set a region of interest used for measuring heartbeat information in a cross section of the heart of a fetus. A further object of the present invention is to optimize at least one of a position and a size of the region of interest that is set on the cross section of the heart of the fetus and used for measuring heartbeat information.

### Solution to Problem

An ultrasound diagnostic device according to the present invention includes a frame sequence generation unit that generates a frame sequence based on a signal acquired by transmitting and receiving an ultrasound wave to and from a periodically moving organ, a candidate region group setting unit that sets a group of candidate regions for each frame in the frame sequence, a correlation value calculation unit that successively calculates, for each of the candidate regions, correlation values between a reference frame and remaining frames other than the reference frame in the frame sequence to generate a correlation value waveform representing a change in the correlation values with respect to time for each of the candidate regions, a stable waveform portion localization unit that localizes a stable waveform portion in the correlation value waveform for each of the candidate regions, an optimum stable waveform portion determination unit that determines an optimum stable waveform portion from among the stable waveform portions respectively localized in the correlation value waveforms which are generated by the correlation value calculation unit, and a period information calculation unit that calculates period information on a motion of the organ.

According to the above-described configuration, the correlation value waveforms, each of which corresponds to one of the candidate regions, are generated, and the stable waveform portion is determined for each of the correlation value waveforms. In other words, an object to be evaluated is not the whole of each correlation value waveform, but the stable waveform portion in each correlation value waveform. In this case, a continuous and less varying portion of the correlation value waveform may be localized as the stable waveform portion. Alternatively, a collection of waveform fragments which are discretely present on the correlation value waveform in a relationship that they do not vary so much from each other may be localized as the stable waveform portion. After the stable waveform portions corresponding to the candidate regions are localized, the optimum stable waveform portion is selected from among the stable waveform portions. This selection corresponds to determination of a stable region (the region of interest used for measurement of period information) in the candidate regions. Therefore, the period information is calculated from the optimum stable waveform portion or from the correlation value waveform including the optimum stable waveform portion. When the organ to be measured is a heart, heartbeat information such as a heart rate is calculated as the period information.

In the above-described configuration, a plurality of the candidate regions which are candidates for the region of interest are prepared, and the correlation value waveforms calculated for the candidate regions are evaluated to select the optimum candidate region (or a waveform portion to be referenced). In this way, the region of interest is determined based on the evaluation of the correlation value waveforms, which leads to an increased degree of accuracy of setting the region of interest. Further, this configuration can solve the problem of complexity in user operation to set the region of interest while predicting or taking into account stability.

As empirical facts, it is rare that the correlation value waveform is stable in its entirety, and in most cases, stable and unstable portions are mixedly present in each correlation value waveform. This tendency is more pronounced especially when the heart of a fetus is measured. According to the present invention, when the correlation value waveform is evaluated, it is possible to evaluate the correlation value waveform from which unstable waveform portions (for example, portions having any excessively extreme value) other than the stable portion are removed. This can facilitate active use of useful or good waveform information. The candidate region associated with the optimum stable waveform portion corresponds to a portion of an organ where the organ moves in a stably periodic manner. Therefore, according to the present invention, period information can be measured with a high degree of accuracy from such a stably periodically moving portion of the organ.

Preferably, the group of candidate regions consists of candidate regions defined so as to have a relationship such that the candidate regions are not identical to each other in the whole or a part of a frame area. In this way, the candidate regions suitable for calculating period information can be defined. The frame sequence consists of frames arranged on a time axis. Each of the frames corresponds to a cross section of a measurement target in an organ, and in particular, corresponds to a beam scan surface or a tomographic image of the organ. The group of candidate regions is defined in the whole or a part of the each of the frames. It is preferable that multiple groups of candidate regions having different patterns are prepared, and one of the groups of candidate regions is defined manually or automatically depending on a site to be diagnosed.

Preferably, the group of candidate regions includes two or more candidate regions that are set at least at different positions in the whole or a part of a frame area. This allows optimization of the position of the region with respect for which period information is calculated.

Preferably, the group of candidate regions includes two or more candidate regions that have at least different sizes in the whole or a part of the frame area. This allows optimization of the size of the region with respect for which period information is calculated.

Preferably, the stable waveform portion localization unit localizes the stable waveform portion through waveform analysis of the correlation value waveforms.

Preferably, the stable waveform portion localization unit includes a generation unit for calculating a provisional period information element at each interval between adjacent peaks in the correlation value waveform to generate a provisional period information sequence, and a determination unit for determining provisional period information elements that satisfy a stability condition in the provisional period information sequence, to localize the stable waveform portion. Because, in this way, it becomes possible to evaluate the correlation value waveform from which any excessively extreme provisional period information elements are removed, the optimum stable waveform portion can be determined without being influenced by the excessively extreme provisional period information elements.

Preferably, the determination unit has a sorting unit for sorting the provisional period information sequence in accordance with a sort condition, and an identifying unit for identifying, as the provisional period information elements in the sorted provisional period information sequence, a specific number of provisional period information elements arranged along a sort direction.

Preferably, the sorting unit sorts the provisional period information sequence in descending or ascending order by value, and the identifying unit identifies a middle part of the sorted provisional period information sequence as the specific number of provisional period information elements. Parts other than the middle part include excessively extreme provisional period information elements, whereas the middle part includes provisional period information elements which are more stable than those in the other parts. Therefore, when a waveform portion corresponding to the provisional period information elements contained in the middle part is determined as the stable waveform portion, it becomes possible to evaluate the correlation value waveform from which the excessively extreme provisional period information elements are eliminated.

Preferably, the determination unit has a function of setting two or more variation reference windows to the provisional period information sequence to calculate variations, and a function of finding a smallest variation in the variations to localize the stable waveform portion in the correlation value waveform. The reference window associated with the smallest variation contains the provisional period information which is more stable than that contained in the other reference windows. According to this configuration, it is possible to evaluate the correlation value waveform from which the excessively extreme provisional period information is eliminated.

Preferably, the optimum stable waveform portion determination unit determines, as the optimum stable waveform portion, the stable waveform portion associated with a variation which is smallest among those in the stable waveform portions. In the candidate region corresponding to the optimum stable waveform portion associated with the smallest variation, periodic motion is more stable than that in the other candidate regions. Therefore, when the period information is acquired based on the correlation value waveform obtained from the candidate region corresponding to the optimum stable waveform portion associated with the smallest variation, the accuracy of measuring period information is improved.

Preferably, the period information calculation unit calculates the period information from the optimum stable waveform portion. The optimum stable waveform portion is more stable than the other waveform portions (free of excessively extreme portions). For this reason, the accuracy of measuring period information can be further improved by obtaining the period information from the optimum stable waveform portion.

Further, an ultrasound image processing method according to the present invention includes a step of receiving a frame sequence generated based on a signal acquired by transmitting and receiving an ultrasound wave to and from a periodically moving organ and setting a group of candidate regions for each frame in the frame sequence, a step of successively calculating correlation values between a reference frame and remaining frames other than the reference frame in the frame sequence to generate a correlation value waveform representing a change in the correlation values with respect to time for each of the candidate regions, a step of localizing a stable waveform portion in the correlation value waveform for each of the candidate regions, a step of determining an optimum stable waveform portion from among the stable waveform portions localized in the generated correlation value waveforms, and a step of calculating period information on a motion of the organ based on the correlation value waveform obtained from one of the candidate regions that corresponds to the optimum stable waveform portion.

### Advantageous Effects of Invention

According to the present invention, the accuracy of measuring period information on a periodically moving organ can be improved in an ultrasound diagnostic device.

### BRIEF DESCRIPTION OF DRAWINGS

FIG. 1 is a block diagram showing an example of an ultrasound diagnostic device according to an embodiment of the present invention;

FIG. 2 is a schematic diagram showing an example for setting a group of candidate regions;

FIG. 3 is a diagram showing an example of each correlation value waveform in the candidate regions;

FIG. 4 is a flowchart showing an operation according to Example 1;

FIG. 5A is a diagram for explaining the operation according to Example 1;

FIG. 5B is a diagram for explaining the operation according to Example 1;

FIG. 6 is a flowchart showing an operation according to Example 2;

FIG. 7A is a diagram for explaining the operation according to Example 2;

FIG. 7B is a diagram for explaining the operation according to Example 2;

FIG. 7C is a diagram for explaining the operation according to Example 2;

FIG. 7D is a diagram for explaining the operation according to Example 2;

FIG. 8A is a schematic diagram showing an example for setting a group of candidate regions according to Modification Example 1;

FIG. 8B is a schematic diagram showing the example for setting the group of candidate regions according to Modification Example 1;

FIG. 8C is a schematic diagram showing the example for setting the group of candidate regions according to Modification Example 1;

FIG. 8D is a schematic diagram showing the example for setting the group of candidate regions according to Modification Example 1;

FIG. 8E is a schematic diagram showing the example for setting the group of candidate regions according to Modification Example 1;

FIG. 8F is a schematic diagram showing the example for setting the group of candidate regions according to Modification Example 1;

FIG. 8G is a schematic diagram showing the example for setting the group of candidate regions according to Modification Example 1;

FIG. 8H is a schematic diagram showing the example for setting the group of candidate regions according to Modification Example 1;

FIG. 8I is a schematic diagram showing the example for setting the group of candidate regions according to Modification Example 1;

FIG. 9A is a schematic diagram showing an example for setting a group of candidate regions according to Modification Example 2;

FIG. 9B is a schematic diagram showing the example for setting the group of candidate regions according to Modification Example 2;

FIG. 9C is a schematic diagram showing the example for setting the group of candidate regions according to Modification Example 2;

FIG. 9D is a schematic diagram showing the example for setting the group of candidate regions according to Modification Example 2;

FIG. 9E is a schematic diagram showing the example for setting the group of candidate regions according to Modification Example 2;

FIG. 9F is a schematic diagram showing the example for setting the group of candidate regions according to Modification Example 2;

FIG. 10 is a schematic diagram showing an example for setting a group of candidate regions according to Modification Example 3;

FIG. 11A is a schematic diagram showing the example for setting the group of candidate regions according to Modification Example 3;

FIG. 11B is a schematic diagram showing the example for setting the group of candidate regions according to Modification Example 3;

FIG. 11C is a schematic diagram showing the example for setting the group of candidate regions according to Modification Example 3;

FIG. 11D is a schematic diagram showing the example for setting the group of candidate regions according to Modification Example 3;

FIG. 11E is a schematic diagram showing the example for setting the group of candidate regions according to Modification Example 3;

FIG. 11F is a schematic diagram showing the example for setting the group of candidate regions according to Modification Example 3;

FIG. 11G is a schematic diagram showing the example for setting the group of candidate regions according to Modification Example 3;

FIG. 11H is a schematic diagram showing the example for setting the group of candidate regions according to Modification Example 3;

FIG. 11I is a schematic diagram showing the example for setting the group of candidate regions according to Modification Example 3;

FIG. 11J is a schematic diagram showing the example for setting the group of candidate regions according to Modification Example 3;

FIG. 11K is a schematic diagram showing the example for setting the group of candidate regions according to Modification Example 3, and

FIG. 11L is a schematic diagram showing the example for setting the group of candidate regions according to Modification Example 3.

### DESCRIPTION OF EMBODIMENT

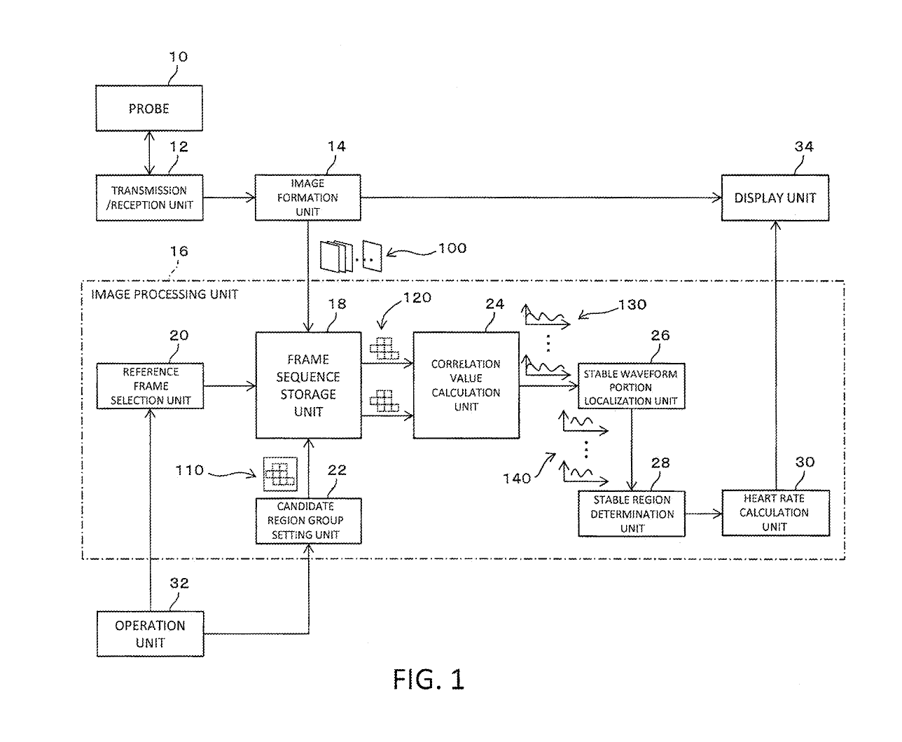

FIG. 1 shows an example of an ultrasound diagnostic device according to an embodiment of the present invention. The ultrasound diagnostic device is a device installed in a medical institution such as a hospital and is used for forming ultrasound images from ultrasound waves transmitted to and received from a human body. The ultrasound diagnostic device according to this embodiment has, as described in detail below, a function of transmitting and receiving the ultrasound waves to and from a fetus to measure heartbeat information of the fetus. An object to be measured with the device may be another organ that moves periodically.

In FIG. 1, a probe 10 is a transducer that transmits and receives ultrasound waves to and from a diagnosis region including a target object. The probe 10 is equipped with vibration elements that transmit and receive the ultrasound waves. The vibration elements are configured to form ultrasound beams. The ultrasound beams are electronically scanned repetitively. This repetitive scanning successively forms beam scanned surfaces. As electronic scanning methods, electronic sector scanning, electronic linear scanning, and other scanning schemes have been known.

A transmission/reception unit 12 outputs, in transmitting operation, delayed transmission signals to the vibration elements equipped in the probe 10. This causes the vibration elements to send transmission beams into a biological body. In receiving operation, upon receipt of reflected waves from the biological body, the vibration elements output reception signals to the transmission/reception unit 12. In the transmission/reception unit 12, the reception signals are processed through phasing addition and other operation to form reception beams. In other words, the transmission/reception unit 12 outputs phased and added reception signals (beam data). The transmission and reception beams, which collectively constitute an ultrasound beam, are electronically scanned by the action of the transmission/reception unit 12. In this way, the above-described beam scanned surfaces are formed. Each of the beam scanned surfaces correspond to multiple beam data elements that constitute a reception frame (reception frame data). Each beam data element is composed of echo data elements arranged along a depth direction. Multiple reception frames arranged on a time axis are output from the transmission/reception unit 12 by repeating operation of electronically scanning the ultrasound beams. The output reception frames constitute a reception frame sequence. The beam data output from the transmission/reception unit 12 is sent via an unillustrated signal processing unit to an image formation unit 14. The signal processing unit includes a detection circuit, a logarithmic compression circuit, and other components. It should be noted that a technique such as transmission aperture synthesis may be used in transmission and reception of the ultrasound waves.

The image formation unit 14 is composed of a digital scan converter having a coordinate converting function, an interpolation processing function, and other functions. The image formation unit 14 forms, based on the reception frame sequence, a display frame sequence 100 consisting of a plurality of display frames. Each of the display frames constituting the display frame sequence 100 is data of a B mode tomographic image. The display frame sequence 100 is output to a display unit 34 such as a monitor and displayed thereon. In this way, the B mode tomographic images are displayed as a moving picture in real time. In this embodiment, the display frame sequence 100 is stored in a frame sequence storage unit 18.

An image processing unit 16 includes the frame sequence storage unit 18, a reference frame selection unit 20, a candidate region group setting unit 22, a correlation value calculation unit 24, a stable waveform portion localization unit 26, a stable region determination unit 28, and a heart rate calculation unit 30.

The reference frame selection unit 20 selects, from the display frame sequence 100 stored in the frame sequence storage unit 18, a reference frame used as a criterion of correlation value calculation. The reference frame selection unit 20 selects the reference frame, for example, in accordance with user operation input through an operation unit 32. For example, the display frame sequence 100 stored in the frame sequence storage unit 18 is displayed by the display unit 34. A user specifies the reference frame through the operation unit 32 while viewing the display frame sequence 100 displayed on the display unit 34. Alternatively, the reference frame selection unit 20 may select as the reference frame an arbitrary display frame in the display frame sequence 100. Selection of the reference frame may be automated. For example, the reference frame may be specified by image analysis.

The candidate region group setting unit 22 sets a candidate region group 110 to each display frame in the display frame sequence to be processed. For example, the candidate region group 110 is composed of candidate regions dispersedly defined to have a relationship such that they are not identical to each other. Specifically, the candidate region group setting unit 22 may set the candidate regions in different positions for each display frame in the display frame sequence. Further, the candidate region group setting unit 22 may set the candidate regions which differ in size from each other. In the present embodiment, the candidate region group setting unit 22 sets the candidate region group 110 to the heart of a fetus in each of the display frames in the display frame sequence. The candidate region group setting unit 22 sets the candidate region group 110, for example, in accordance with user operation input from the operation unit 32. For example, the reference frame is displayed by the display unit 34. While viewing the reference frame displayed on the display unit 34, a user uses the operation unit 32 to specify a position where the candidate region group 110 is set. The candidate region group 110 is set at the specified position. The candidate region group setting unit 22 sets the candidate region group 110 in each of the display frames constituting the display frame sequence to be processed, at the same position as that specified to the reference frame. Alternatively, the candidate region group setting unit 22 may perform image analysis of the reference frame to set the candidate region group in a region of the heart of a fetus. The candidate region group setting unit 22 reads, from the frame sequence storage unit 18, a display frame sequence 120 corresponding to each candidate region and outputs the display frame sequences 120 to the correlation value calculation unit 24.

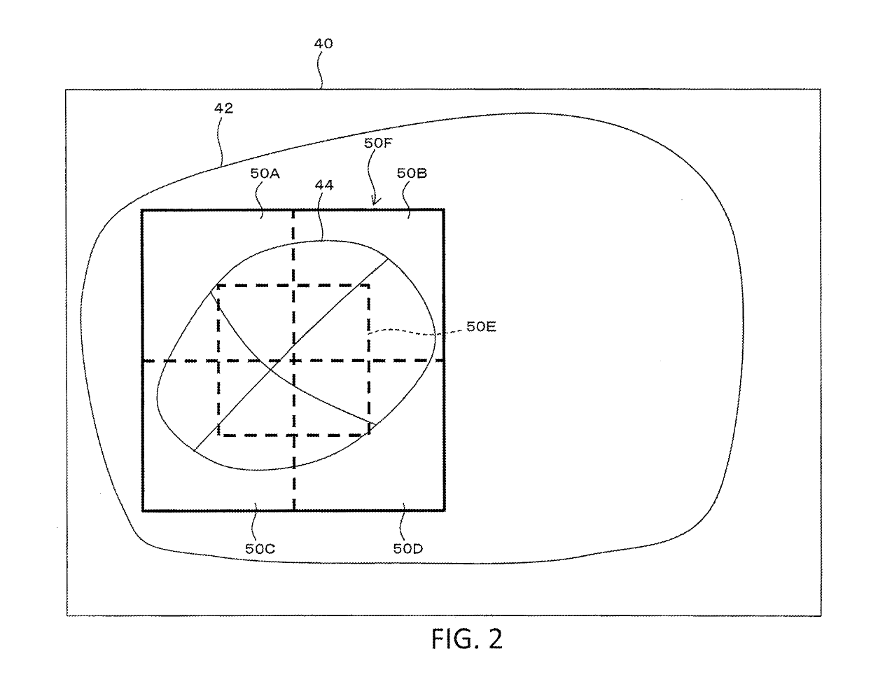

FIG. 2 shows an example of setting the candidate region group. In a reference frame 40, a body 42 and a heart 44 of the fetus are shown. In the example of FIG. 2, six rectangular candidate regions (candidate regions 50A to 50F) are defined. The candidate regions 50A to 50E are set on different positions in such a manner that each of the candidate regions 50A to 50E includes a part of the heart 44. The candidate region 50F is set so as to include the whole of the heart 44. The candidate regions 50A to 50E are all of the same size. The candidate regions 50A to 50D are set without overlapping each other on quarter regions obtained by dividing the candidate region 50F into 4 equal parts. The candidate region 50E is set so as to overlap all of the candidate regions 50A to 50F. Although in the example of FIG. 2 the candidate regions 50A to 50F have the geometry of a rectangle, the geometry may be a polygon other than the rectangle, a circle, or an ellipse. Further, the candidate regions 50A to 50E may be of the same size or may be different in size. The candidate regions 50A to 50D may be defined to overlap each other. Still further, the number of candidate regions is not limited to that shown in the example of FIG. 2, and any number of candidate regions may be set so long as the number is greater than one. The geometry, size, number, and setting position of the candidate regions may be arbitrarily selected, and may be specified by the user operation through the operation unit 32.

Referring back to FIG. 1, the correlation value calculation unit 24 successively calculates, for each of the candidate regions, correlation values between the reference frame and remaining display frames other than the reference frame. This generates a correlation value waveform 130 representing a change in the correlation values with respect to time for each of the candidate regions. A specific example is illustrated. It is assumed that display frames F1, F2, F3, and F4 are the display frames to be processed. In this example, the correlation value calculation unit 24 calculates, for each of the candidate regions, a correlation value between the reference frame (for example, the display frame F1) and the display frame F2, a correlation value between the reference frame and the display frame F3, and a correlation value between the reference frame and the display frame F4. As a result, the correlation value waveform representing the change in the correlation value with respect to time is obtained for each of the candidate regions. As the correlation value, a publicly-known technique, such as the sum of square differences (SSD), the sum of absolute differences (SAD), or an average value difference, may be used.

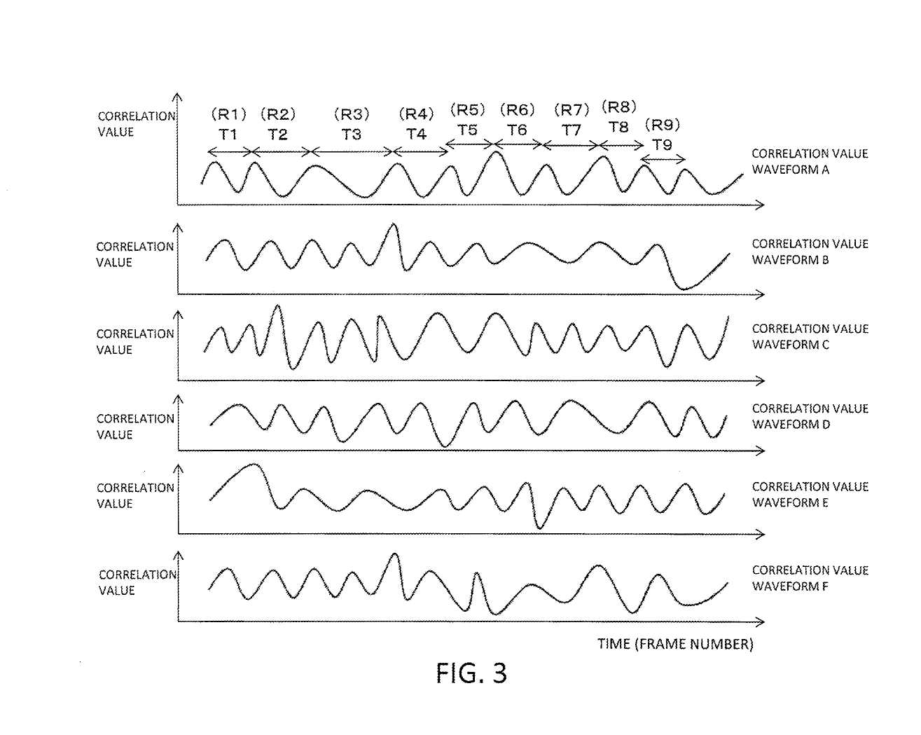

FIG. 3 shows an example of the correlation value waveforms corresponding to the candidate regions 50A to 50F. In FIG. 3, the horizontal axis is a time axis, and the vertical axis represents the correlation values. A correlation value waveform A is a waveform showing a change in the correlation value with respect to time in the candidate region 50A illustrated in FIG. 2. A correlation value waveform B shows a change in the correlation value with respect to time in the candidate region 50B. A correlation value waveform C represents a change in the correlation value with respect to time in the candidate region 50C. A correlation value waveform D represents a change in the correlation value with respect to time in the candidate region 50D. A correlation value waveform E represents a change in the correlation value with respect to time in the candidate region 50E. A correlation value waveform F represents a change in the correlation value with respect to time in the candidate region 50F.

Referring back to FIG. 1, the stable waveform portion localization unit 26 localizes a stable waveform portion 140 in the correlation value waveform 130 for each of the candidate regions. Specifically, the stable waveform portion localization unit 26 excludes a portion which contains an excessively extreme value from the correlation value waveform 130 for each of the candidate regions to localize the stable waveform portion 140 where the waveform is stable. For example, the stable waveform portion localization unit 26 calculates provisional heart rates of the heart based on the correlation value waveform 130 for each of the candidate regions, and localizes the stable waveform portion 140 from the correlation value waveform 130 based on the provisional heart rates.

The stable region determination unit 28 compares the stable waveform portions 140 localized in the correlation value waveforms 130 with each other to determine an optimum stable waveform portion among the stable waveform portions 140. Then, the stable region determination unit 28 determines a candidate region corresponding to the optimum stable waveform portion as a stable region. For each of the candidate regions, the stable region determination unit 28 calculates, for example, a variation of the provisional heart rates corresponding to the stable waveform portion 140 to localize a stable waveform portion (the optimum stable waveform portion) having the smallest variation of the provisional heart rates, and determines, as the stable region, the candidate region corresponding to the optimum stable waveform portion.

The heart rate calculation unit 30 calculates, based on the correlation value waveform obtained from the stable region, heartbeat information of the fetus. The heartbeat information may be, for example, the heart rate.

It should be noted that components other than the probe 10 illustrated in FIG. 1 may be implemented by means of hardware resources, such as, for example, a processor or an electronic circuit, and a device, such as a memory, may be used for the implementation when necessary. Alternatively, the components other than the probe 10 may be implemented by, for example, a computer. In other words, hardware resources contained in the computer, such as a CPU, a memory, and a hard disc, may cooperate with software (a program) that specifies operation of the CPU and other devices to implement all or some of the components other than the probe 10. The program may be stored via a recording medium, such as a CD or a DVD, or through a communication channel, such as a network, into an unillustrated storage device. As another example, the components other than the probe 10 may be implemented by a DSP (digital signal processor), a FPGA (field programmable gate array), or the like.

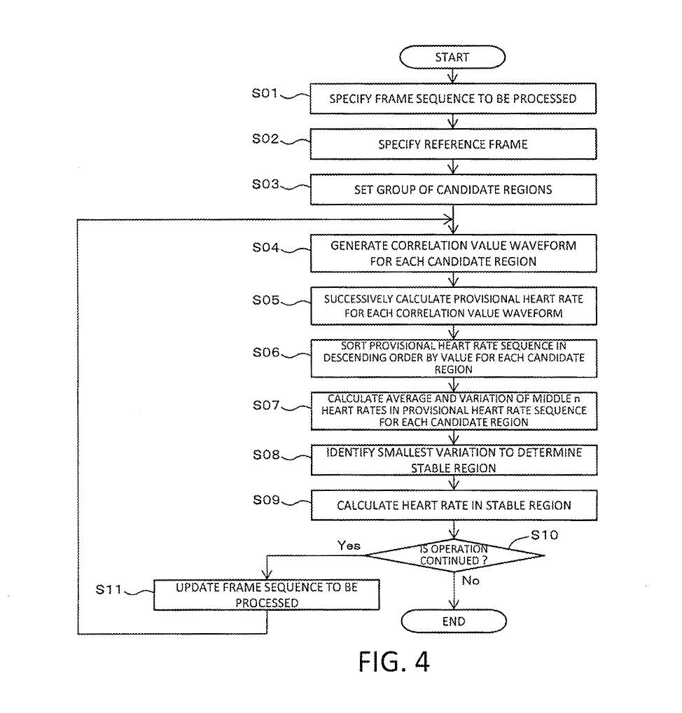

Next, referring to a flowchart shown in FIG. 4, Example 1 of an operation performed by the ultrasound diagnostic device according to this embodiment will be described. First, the display frame sequences stored in the frame sequence storage unit 18 are displayed on the display unit 34. The user specifies, through the operation unit 32, a target frame sequence to be processed from among the display frame sequences (S01). Then, the user uses the operation unit 32 to specify the reference frame in the target frame sequence to be processed (S02), and subsequently displays the reference frame on the display unit 34. The user specifies, by means of the operation unit 32, a setting position of a group of candidate regions while viewing the reference frame. This causes the candidate region group setting unit 22 to set the group of candidate regions on each frame in the target frame sequence to be processed (S03). As an example, the candidate region group setting unit 22 sets the candidate regions 50A to 50F as shown in FIG. 2 on each frame in the target frame sequence to be processed. After the group of candidate regions is set, the correlation value calculation unit 24 successively calculates, for each of the candidate regions, correlation values between the reference frame and remaining display frames, to generate the correlation value waveform for each of the candidate regions (S04). For example, as shown in FIG. 3, the correlation value calculation unit 24 generates the correlation value waveforms A to F for the candidate regions 50A to 50F.

Then, the stable waveform portion localization unit 26 successively calculates the provisional heart rates for each of the correlation value waveforms (S05). Specifically, the stable waveform portion localization unit 26 successively searches for peak points (local maximum or minimum points) in the correlation value waveform for each of the candidate regions, and calculates each time interval between peak points (local maximum or minimum points) adjacent to each other as a provisional one-heartbeat time period. Next, based on two or more provisional one-heartbeat time periods, the stable waveform portion localization unit 26 calculates per-unit-time provisional heart rates (bmp) for each of the candidate regions. In this way, the provisional heart rates arranged on the time axis are calculated, to constitute a provisional heart rate sequence. Referring to FIG. 3, the stable waveform portion localization unit 26 calculates, for the correlation value waveform A, each of time intervals T1 to T9 between the adjacent peak points (for example, local maximum values) as the provisional one-heartbeat time period. Then, the stable waveform portion localization unit 26 calculates, from the time intervals T1 to T9, the per-unit-time provisional heart rates R1 to R9. The provisional heart rates R1 to R9 arranged on the time axis constitute the provisional heart rate sequence. The stable waveform portion localization unit 26 similarly calculates the provisional heart rate sequences for the correlation value waveforms B to F.

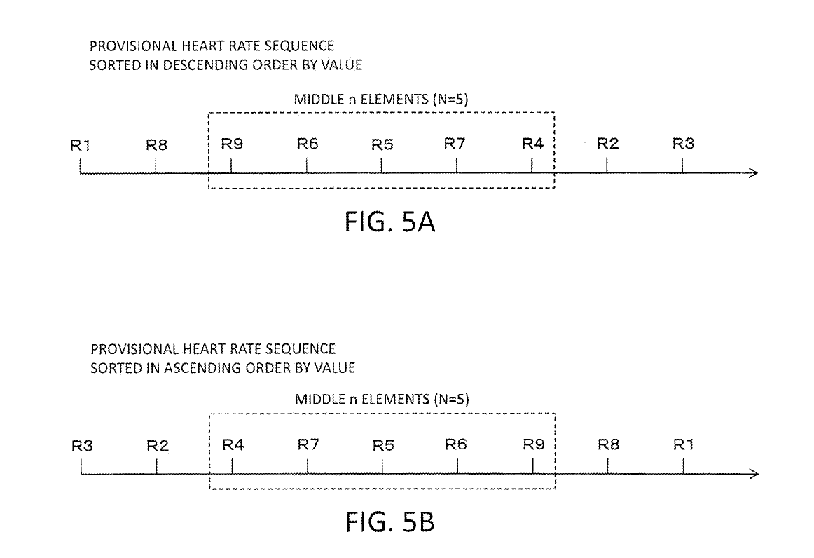

Then, the stable waveform portion localization unit 26 sorts (rearranges), for each of the candidate regions, the provisional heart rate sequence in descending order by value (S06). Referring to the example of the provisional heart rates R1 to R9, the stable waveform portion localization unit 26 sorts as shown in FIG. 5A the provisional heart rates R1 to R9 in descending order by value. Alternatively, as shown in FIG. 5B, the stable waveform portion localization unit 26 may sort the provisional heart rates R1 to R9 in ascending order by value. The stable waveform portion localization unit 26 also sorts the provisional heart rate sequences for the correlation value waveforms B to F.

Next, the stable waveform portion localization unit 26 calculates an average value and a variation of middle N elements (N elements positioned on a middle part) in the sorted provisional heart rate sequence for each of the candidate regions (S07). Reference letter N represents an integer. Assuming, for example, N=5 in the example shown in FIG. 5A, the stable waveform portion localization unit 26 calculates an average value and a variation of five provisional heart rates (provisional heart rates R9, R6, R5, R7, and R4) placed in the middle part. Alternatively, as shown in FIG. 5B, the stable waveform portion localization unit 26 may calculate an average value and a variation of the middle N elements in the provisional heart rates R1 to R9 which are sorted in ascending order by value. The stable waveform portion localization unit 26 further calculates each average value and each variation of the middle N elements in the sorted provisional heart rate sequences for the correlation value waveforms B to F. Note that although the number of elements is defined as N=5 in the examples shown in FIGS. 5A and 5B, any integer other than 5 may be used for N.

Here, the variation will be explained. When each value of the N provisional heart rates is defined as xᵢ (i=1 to N) and an average of the N provisional heart rates is defined as m, a variance is obtained by equation (1) as follows.

$$\begin{matrix}[ Equation 1 ] & \\ {σ}^{2} = \frac{1}{N} {\sum }_{i = 1}^{N} {( {x}_{i} - m )}^{2} & ( 1 )\end{matrix}$$

A positive square root σ of the variance is referred to as a standard deviation.

Further, a value obtained by dividing the standard deviation σ by the average m is referred to as a coefficient of variation (CV). The coefficient of variation CV is expressed by equation (2) as follows.

CV=σ/m (2)

The coefficient of variation CV represents a relative variation that does not depend on the average. The standard deviation σ can have the same value of, for example, 20 in cases where the average value is 50 and where the average value is 200. It can be considered that in the latter case (average value=200), the variation is smaller (CV=0.4 in the case where the average value is 50, whereas CV=0.1 in the case where the average value is 200). The stable waveform portion localization unit 26 calculates the variations CVs of the middle N provisional heart rates in the correlation value waveforms A to F.

Then, the stable region determination unit 28 compares the variations CVs in the correlation value waveforms A to F with each other to identify a correlation value waveform having the smallest variation CV, and determines a candidate region (the stable region) corresponding to the identified correlation value waveform (S08). Specifically, the stable region determination unit 28 evaluates degrees of stability of the correlation value waveforms A to F based on the variations CVs of the middle N elements in the provisional heart rate sequences to find a correlation value waveform having the highest degree of stability (the smallest variation CV). Assuming, as one example, that the variation CV of the correlation value waveform A is smallest among those of the correlation value waveforms A to F, the stable region determination unit 28 determines the candidate region 50A corresponding to the correlation value waveform A as the stable region.

Alternatively, the stable waveform portion localization unit 26 may calculate the standard deviation σ of the middle N elements in the provisional heart rate sequence for each of the correlation value waveforms to localize a correlation value waveform having the smallest standard deviation σ, and determine a candidate region corresponding to that correlation value waveform as the stable region.

After the stable region is determined as described above, the heart rate calculation unit 30 calculates the heart rate in the stable region (S09). For example, the heart rate calculation unit 30 calculates, as the heart rate (bpm) of the fetus, an average value of the provisional heart rate sequence (all provisional heart rates) in the stable region. Instead, the heart rate calculation unit 30 may calculate the average value of the middle N elements in the sorted provisional heart rate sequence as the heart rate of the fetus. The heart rate of the fetus is output, for example, to the display unit 34 and displayed thereon. For example, when the candidate region 50A is determined as the stable region, the heart rate calculation unit 30 calculates, as the heart rate of the fetus, the average value of the middle N elements in the sorted provisional heart rate sequence (for example, the average value of the provisional heart rates R9, R6, R5, R7, and R4 shown in FIG. 5A).

When the operation is continued (YES is selected in S10), the target display frame sequence to be processed is updated (S11), and operations in steps S04 to S09 are performed on the updated display frame sequence. For example, when the user specifies, through the operation unit 32, a display frame sequence acquired in another time band as an object to be processed, operations in steps S04 to S09 are performed on the specified display frame sequence. On the other hand, when the operation is not continued (NO is selected in S10), measurement of the heart rates is finished.

As described above, in Example 1, the provisional heart rate sequence for each of the candidate regions is sorted in descending or ascending order by value, and the variation CV of the middle N elements in the sorted provisional heart rate sequence is calculated. Then, the correlation value waveform for each of the candidate regions is evaluated based on the variation CV obtained for the each of the candidate regions. In this way, unstable waveform portions (portions having an excessively extreme hart rate) which are not specified as the stable waveform portions can be eliminated from the correlation value waveform to evaluate the correlation value waveform. As a result, it becomes possible to determine the stable region from which the correlation value waveform that is more stable than those in the other candidate regions can be obtained, without being affected by the unstable waveform portions.

In this regard, more detailed description is provided below. The correlation value waveform can inevitably contain abnormal values (excessively extreme heart rates). For this reason, when the correlation value waveform is evaluated based on the variation CV of all of the provisional heart rates contained in the provisional heart rate sequence, the abnormal values are taken into account in the evaluation. In this case, the accuracy of evaluation will be decreased. In contrast to this, the provisional heart rate sequence is, in Example 1, sorted in descending or ascending order by value, and the variation CV of the middle N elements in the sorted provisional heart rate sequence is calculated. Then, the correlation value waveform is evaluated based on the thus-calculated variation CV. When the sorting is performed, the excessively extreme provisional heart rates are allocated to ranges other than a range of the middle N elements. As compared with the other ranges, the range of middle N elements is free of the excessively extreme provisional heart rates. For this reason, the waveform portion corresponding to the middle N provisional hart rates is more stable than those corresponding to the provisional heart rates other than the middle N provisional heart rates, and is accordingly regarded as the stable waveform portion in the correlation value waveform. Therefore, when the variation CV of the middle N elements in the sorted heart rate sequence is used, it becomes possible to evaluate the correlation value waveform from which the abnormal values are excluded, and accordingly determine the stable region. In Example 1, due to the sorting of the provisional heart rate sequence, the stable waveform portion is a collection of waveform portions which are not necessarily continuous on the time axis.

A periodic motion in the stable region is more steady than those in the other candidate regions. Therefore, the accuracy of measuring the heart rate can be improved by calculating the heart rate based on the correlation value waveform obtained from the stable region. Further, the sorted middle N provisional heart rates correspond to the stable waveform portion. Accordingly, when the heart rate is calculated from the stable waveform portion, further improvement in the accuracy of measuring the heart rate can be ensured.

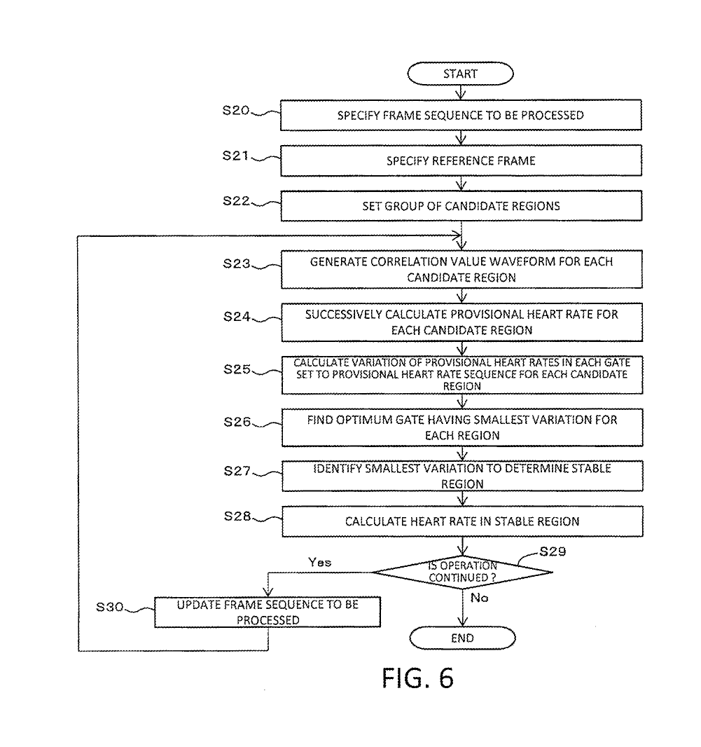

Next, with reference to a flowchart shown in FIG. 6, an operation performed in Example 2 by the ultrasound diagnostic device according to the present embodiment will be described. In Example 2, without sorting the provisional heart rate sequence, calculation is performed to obtain a variation CV of successive N elements which are chronologically arranged in the provisional heart rate sequence. Then, based on the variation CV of successive N elements, the stable region is determined. Process steps according to Example 2 will be described in detail below.

As in the case of Example 1, the user specifies a target frame sequence to be processed from among the display frame sequences stored in the frame sequence storage unit 18 (S20), and further specifies the reference frame in the target frame sequence to be processed (S21). Then, the candidate region group setting unit 22 sets a group of candidate regions on each frame in the target frame sequence to be processed (S22). Then, the stable waveform portion localization unit 26 generates the correlation value waveform for each of the candidate regions (S23), and calculates the provisional heart rate sequence for each of the generated correlation value waveforms (S24). The provisional heart rate sequence is composed of a plurality of provisional heart rates chronologically arranged on the time axis. For example, the candidate regions 50A to 50F are defined as shown in FIG. 2, and the correlation value waveforms A to F and the provisional heart rate sequences corresponding to the candidate regions 50A to 50F are calculated as shown in FIG. 3.

Next, the stable waveform portion localization unit 26 sets a gate (a time window) to the provisional heart rate sequence for each of the candidate regions. The gate includes chronologically arranged successive N provisional heart rates. Then, the stable waveform portion localization unit 26 shifts the gate along a chronological direction to calculate an average value and a variation CV of the successive N provisional heart rates covered under each shifted gate (S25). In other words, the stable waveform portion localization unit 26 calculates a moving average and the variation CV of the provisional heart rate sequence for each of the candidate regions. Then, the stable waveform portion localization unit 26 localizes, for each of the candidate regions, an optimum gate in which the smallest variation CV is obtained (S26). The stable waveform portion localization unit 26 outputs the average value and the variation CV of the successive N provisional heart rates covered under the optimum gate to the stable region determination unit 28.

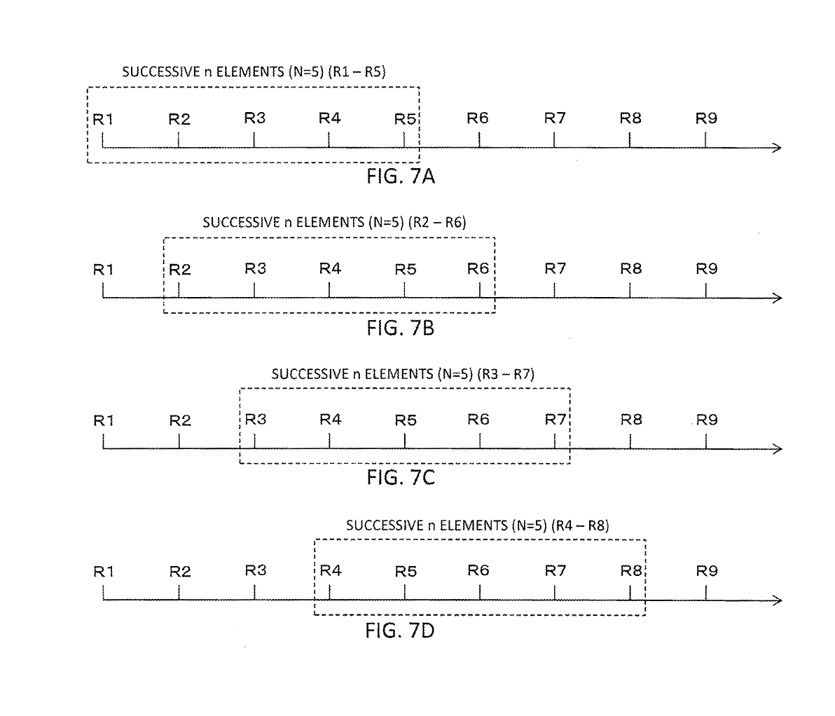

A specific example of operation in steps S25 and S26 is explained with reference to FIGS. 7A, 7B, 7C and 7D. The example operation is explained using, by way of illustration, the provisional heart rates R1 to R9 obtained from the correlation value waveform A shown in FIG. 3. Assuming, for example, that N=5 as shown in FIG. 7A, the stable waveform portion localization unit 26 sets the gate to cover the chronologically arranged provisional heart rates R1 to R5, and calculates an average value and a variation CV of the provisional heart rates R1 to R5. Subsequent to this, the stable waveform portion localization unit 26 shifts, as shown in FIG. 7B, the gate to cover the provisional heart rates R2 and R6, and calculates an average value and a variation CV of the provisional heart rates R2 to R6. Further, the stable waveform portion localization unit 26 calculates an average value and a variation CV of the provisional heart rates R3 to R7 as shown in FIG. 7C, and calculates an average value and a variation CV of the provisional heart rates R4 to R8 as shown in FIG. 7D. Following this, the stable waveform specification unit 26 similarly calculates the moving average and the variation CV of the provisional heart rate sequence. Then, the stable waveform portion localization unit 26 localizes, from among the gates, the optimum gate having the smallest variation CV in the correlation value waveform A, and outputs the average value and the variation CV of the successive N provisional heart rates covered under the optimum gate to the stable region determination unit 28. In the correlation value waveform A, for example, when the variation CV of the provisional heart rates R1 to R5 shown in FIG. 7A is smallest, the stable waveform portion localization unit 26 outputs the average value and variation CV of the provisional heart rates R1 to R5 to the stable region determination unit 28.

The stable waveform portion localization unit 26 identifies an optimum gate having the smallest variation CV for each of the correlation value waveforms A to F, and outputs the average value and variation CV of the successive N provisional heart rates covered under the optimum gate to the stable region determination unit 28. Note that although N=5 is assumed in the example shown in FIGS. 7A to 7D, any value other than 5 may be used for N.

Then, the stable region determination unit 28 identifies, from among the correlation value waveforms A to F, a correlation value waveform associated with the optimum gate having the smallest variation CV of the successive N provisional heart rates, and determines the candidate region (as the stable region) corresponding to the identified correlation value waveform (S27). In other words, the stable region determination unit 28 uses the variation CVs of the successive N elements in the provisional heart rate sequences to evaluate the degrees of stability of the correlation value waveforms A to F, and determines the correlation value waveform having the highest degree of stability (the smallest variation CV). Assuming, for example, that the variation CV of the correlation value waveform A is smallest among the variations CVs of the correlation value waveforms A to F, the stable region determination unit 28 determines the candidate region 50A corresponding to the correlation value waveform A as the stable region.

Alternatively, the stable waveform portion localization unit 26 may calculate a standard deviation a for each gate of the correlation value waveforms, identify the correlation value waveform corresponding to a gate having the smallest standard deviation σ, and determine the candidate region corresponding to the identified correlation value waveform as the stable region.

After the stable region is determined as described above, the heart rate calculation unit 30 calculates the heart rate with respect to the stable region (S28). For example, the heart rate calculation unit 30 calculates an average value of the provisional heart rate sequence (all of the provisional heart rates) in the stable region as the heart rate of the fetus. Instead, the heart rate calculation unit 30 may calculate, as the heart rate of the fetus, an average value of successive N elements covered under the optimum gate in the provisional heart rate sequence for the stable region. The heart rate of the fetus is output, for example, to the display unit 34 and displayed thereon. Assuming, by way of illustration, that the candidate region 50A is specified as the stable region, the heart rate calculation unit 30 calculates, as the heart rate of the fetus, the average value of the successive N elements (such as, for example, the provisional heart rates R1 to R5 shown in FIG. 7A) covered under the optimum gate in the correlation value waveform A.

When operation is continued (YES in S29), the target display frame sequence to be processed is updated (S30), and operation in steps from S23 to S28 is performed on the updated display frame sequence. When operation is not continued, (NO S29), the heart rate measurement is finished.

As described above, in Example 2, the optimum gate covering the successive N elements whose variation CV is smallest in the provisional heart rate sequence is identified for each of the candidate regions. Then, the correlation value waveform in each of the candidate regions is evaluated based on the variation CV of the provisional heart rates covered under the optimum gate for the each of the candidate regions. In this way, it becomes possible to evaluate the correlation value waveforms from which unstable waveform portions are eliminated. As a result, the stable region can be determined without the influence exerted by the unstable waveform portions.

This will be described in detail below. The variation CV of the successive N elements covered under the optimum gate is smaller than those of the successive N elements under other gates. That is, as compared to the other gates, the optimum gate is free of excessively extreme provisional heart rates. For this reason, the waveform portion corresponding to the provisional heart rates under the optimum gate is more stable than the waveform portions corresponding to the provisional heart rates under the other gates, and may be considered to be the stable waveform portion in the correlation value waveform. Accordingly, when the variation CV of the successive N provisional heart rates covered under the optimum gate is used, the correlation value waveform from which the abnormal values are eliminated can be evaluated to determine the stable region.

Then, the accuracy of measuring the heart rate can be improved by calculating the heart rate based on the correlation value waveform obtained from the stable region. Further, the successive N provisional heart rates covered under the optimum gate correspond to the stable waveform portion. The calculation of the heart rate from the stable waveform portion can further ensure improvement in the accuracy of measuring the heart rate.

Meanwhile, according to this embodiment, when the candidate regions are set at different positions, it becomes possible to find, from among the group of the candidate regions, the position of a candidate region suitable for use in calculation of the heart rate (the candidate region where motion is stably periodic). Moreover, when the candidate regions that vary in size are defined, it becomes possible to find, from among the group of the candidate regions, the size of a candidate region suitable for use in calculation of the heart rate.

Still further, because the stable region determination unit 28 determines the stable region, the user is freed from the burden of performing complicated operation to specify the stable region.

Note that Examples 1 and 2 may be combined. For example, the stable region may be determined through the operation according to Example 1, and the heart rate may be calculated through the operation according to Example 2. Specifically, as in the case of Example 1, the stable waveform portion localization unit 26 sorts each provisional heart rate sequence of the candidate regions in descending or ascending order by value, and calculates the variation of the middle N elements in the sorted provisional heart rate sequence. The stable region determination unit 28 determines, as the stable region, the candidate region associated with the smallest variation. The heart rate calculation unit 30 sets the gates to the provisional heart rate sequence in the stable region, and calculates, while shifting the gates, each average value and each variation of N provisional heart rates covered under the gates. Then, the heart rate calculation unit 30 identifies, from among the gates, the optimum gate associated with the smallest variation, and calculates the average value of the N provisional heart rates covered under the optimum gate as the heart rate of the fetus.

Alternatively, the operation according to Example 2 may be used to determine the stable region and the operation according to Example 1 may be used to calculate the heart rate. Specifically, as in the case of Example 2, the stable waveform portion localization unit 26 identifies the optimum gate for each of the candidate regions. The stable region determination unit 28 determines the stable region based on the variation of the provisional heart rates covered under the optimum gate for each of the candidate regions. The heart rate calculation unit 30 sorts the provisional heart rate sequence in the stable region in descending or ascending order by value, and calculates the average value of the middle N elements in the sorted provisional heart rate sequence as the heart rate of the fetus.

As described above, even when Examples 1 and 2 are combined, the heart rate is calculated based on the correlation value waveform obtained from the stable region, which can lead to improved accuracy of measuring the heart rate.

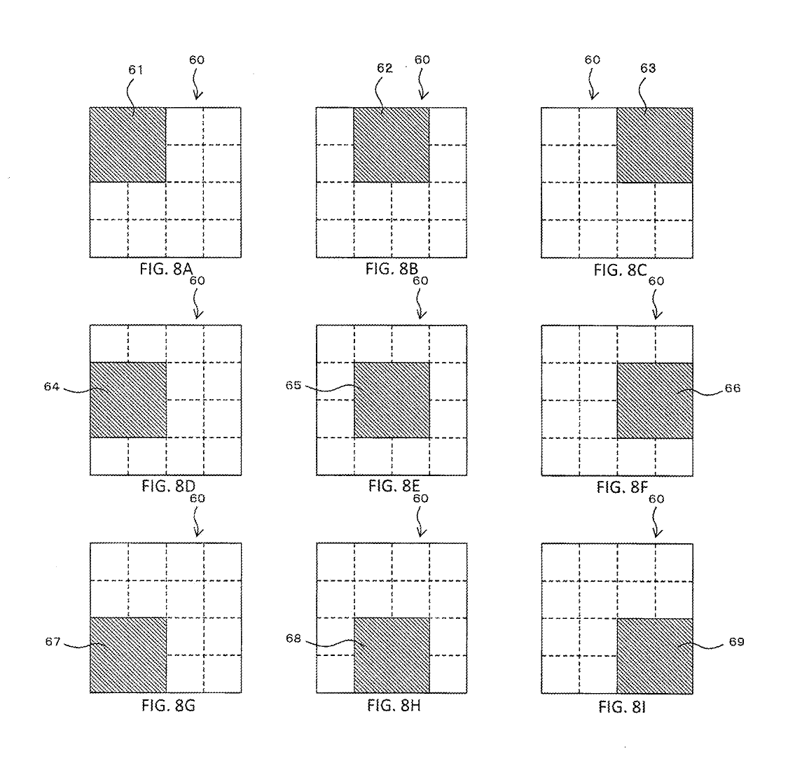

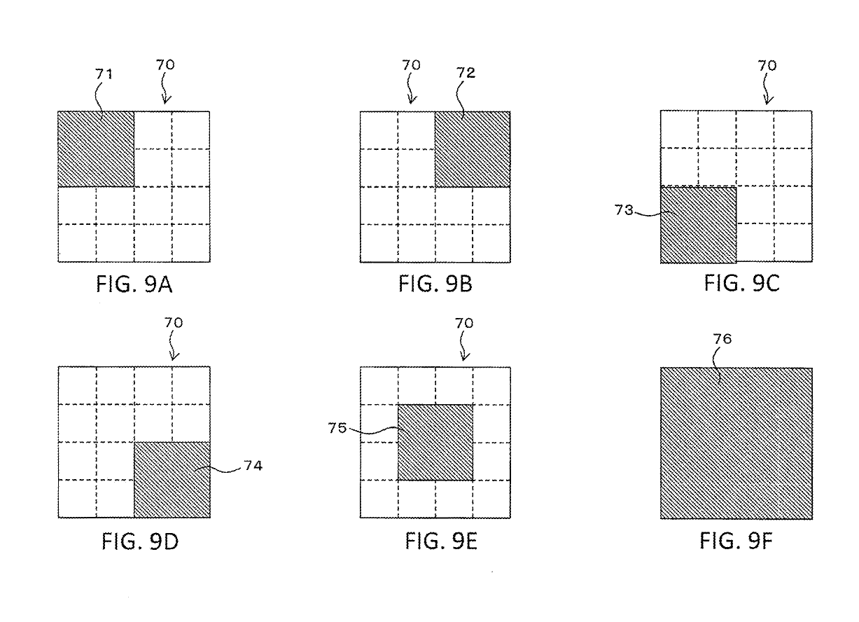

Next, examples of setting the group of candidate regions are described according to modification examples. FIGS. 8A to 8I illustrate the example of setting the group of candidate regions according to Modification Example 1. In Modification Example 1, as shown in FIGS. 8A to 8I nine candidate regions (rectangular candidate regions 61 to 69) having the same geometry and the same size are set in a region of interest 60 defined in the target display frame sequence to be processed. The candidate regions 61 to 69 are set so as to overlap each other in part. FIGS. 9A to 9F illustrate the example of setting the group of candidate regions according to Modification Example 2. In Modification Example 2, as shown in FIGS. 9A to 9F, six candidate regions (rectangular candidate regions 71 to 76) having the same geometry are set in a region of interest 70 defined in the target display frame sequence to be processed. The candidate regions 71 to 75 are equal in size. The candidate region 76 is larger in size than the candidate regions 71 to 75, and defined to cover the whole region of interest 70. It should be noted that the geometry, size, setting position, and the number of candidate regions may be arbitrarily selected, and are not limited to those illustrated in the examples of FIGS. 8A to 8I and FIGS. 9A to 9F.

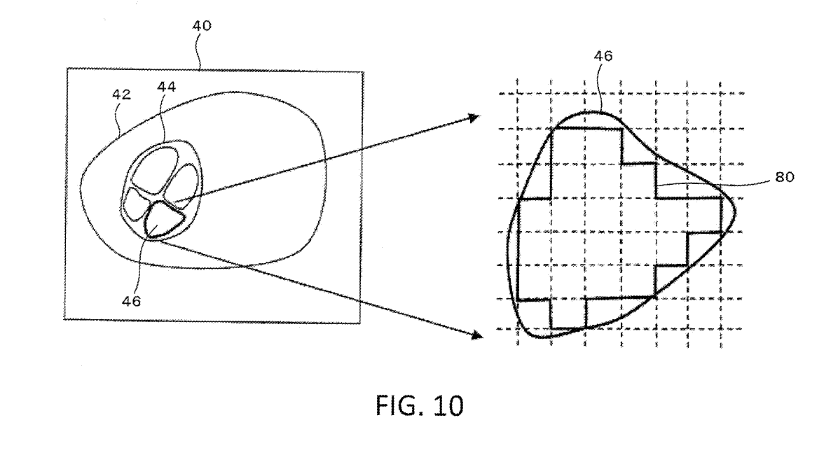

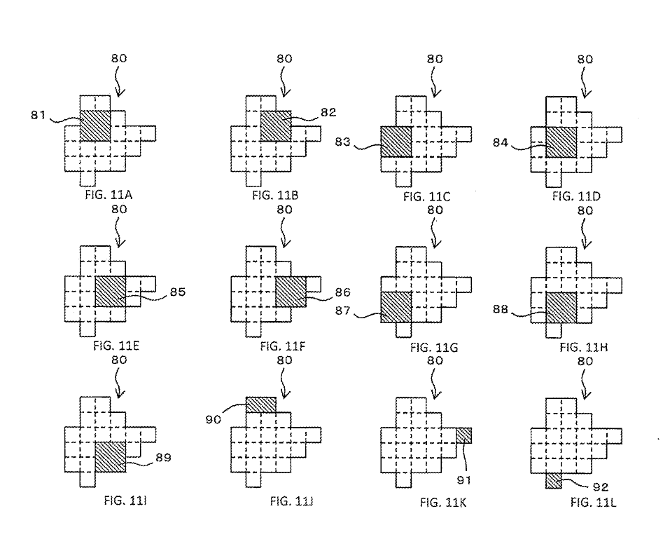

Next, referring to FIG. 10 and FIGS. 11A to 11L, the example of setting the group of candidate regions according to Modification Example 3 is described. As shown in FIG. 10, for example, the candidate region group setting unit 22 performs image analysis operation, such as automatic boundary extraction and a learning function, on the reference frame to automatically find a region of the heart 44 of the fetus or an organ (such as, for example, the left ventricle) inside the heart 44. As one example, the candidate region group setting unit 22 defines the region of interest 46 in a region of the left ventricle and sets the group of candidate regions within the region of interest 46. When rectangular candidate regions are set, for example, the candidate region group setting unit 22 sets, as shown in FIGS. 11A to 11L, the group of candidate regions (candidate regions 81 to 92) within an area 80 which is inscribed in the region of interest 46. The geometry, size, and setting positions of the candidate regions 81 to 92 may be arbitrarily specified. Such automatic finding of the target object and automatic setting of the candidate regions can contribute to saving time and effort of the user to set the candidate regions.

Although in this embodiment the stable waveform portion and the stable region are localized and determined using the provisional heart rates, a plurality of provisional one-heartbeat time periods derivable from the correlation value waveform may be used to localize and determine the stable waveform portion and the stable region.

Further, although in the embodiment the display frame sequence processed through digital scan conversion is utilized to localize the stable waveform portion and determine the stable region, a received frame sequence which is not processed through the digital scan conversion may be utilized to localize the stable waveform portion and determine the stable region. In this case, the received frame sequence output from the transmission/reception unit 12 is stored in the frame sequence storage unit 18. The image processing unit 16 performs processing on the received frame sequence to localize the stable waveform portion, determine the stable region, and calculate the heart rate of the fetus.

### REFERENCE SIGNS LIST

10 probe, 12 transmission/reception unit, 14 image formation unit, 16 image processing unit, 18 frame sequence storage unit, 20 reference frame selection unit, 22 candidate region group setting unit, 24 correlation value calculation unit, 26 stable waveform portion localization unit, 28 stable region determination unit, 30 heart rate calculation unit, 32 operation unit, 34 display unit.

1. An ultrasound diagnostic device comprising:
a frame sequence generation unit that generates a frame sequence based on a signal obtained by transmitting and receiving an ultrasound wave to and from a periodically moving organ;
a candidate region group setting unit that sets a group of candidate regions for each frame in the frame sequence;
a correlation value calculation unit that successively calculates, for each of the candidate regions, correlation values between a reference frame and remaining frames other than the reference frame in the frame sequence, to generate, for each of the candidate regions, a correlation value waveform representing a change in the correlation values with respect to time;
a stable waveform portion localization unit that localizes a stable waveform portion in the correlation value waveform for each of the candidate regions;
an optimum stable waveform portion determination unit that determines an optimum stable waveform portion from among a plurality of the stable waveform portions localized in a plurality of the correlation value waveforms which are generated by the correlation value calculation unit, and
a period information calculation unit that calculates period information on a motion of the organ based on the correlation value waveform obtained from one of the candidate regions that corresponds to the optimum stable waveform portion.

2. The ultrasound diagnostic device according to claim 1, wherein the group of candidate regions consists of a plurality of candidate regions defined so as to have a relationship such that the candidate regions are not identical to each other in the whole or a part of a frame area.

3. The ultrasound diagnostic device according to claim 2, wherein the group of candidate regions includes a plurality of candidate regions that are set at least at different positions in the whole or a part of the frame region.

4. The ultrasound diagnostic device according to claim 2, wherein the group of candidate regions includes a plurality of candidate regions that have at least different sizes in the whole or a part of the frame area.

5. The ultrasound diagnostic device according to claim 1, wherein the stable waveform portion localization unit localizes the stable waveform portion through waveform analysis of the correlation value waveform.

6. The ultrasound diagnostic device according to claim 5, wherein:
the stable waveform portion localization unit comprises;
a generation unit that calculates a provisional period information element at each interval between adjacent peaks in the correlation value waveform to generate a provisional period information sequence, and
a determination unit that determines a plurality of provisional period information elements that satisfy a stability condition in the provisional period information sequence to localize the stable waveform portion.

7. The ultrasound diagnostic device according to claim 6, wherein:
the determination unit comprises;
a sorting unit that sorts the provisional period information sequence according to a sort condition, and
an identifying unit that identifies, as the plurality of provisional period information elements, a specific number of provisional period information elements arranged along a sort direction in the sorted provisional period information sequence.

8. The ultrasound diagnostic device according to claim 7, wherein:
the sorting unit sorts the provisional period information sequence in descending or ascending order by value, and
the identifying unit that identifies the specific number of provisional period information elements from a middle part of the sorted provisional period information sequence.

9. The ultrasound diagnostic device according to claim 6, wherein:
the determination unit has;
a function of setting a plurality of variation reference windows onto the provisional period information sequence to calculate a plurality of variations, and
a function of identifying a smallest variation from among the plurality of variations to localize the stable waveform portion in the correlation value waveform.

10. The ultrasound diagnostic device according to claim 1, wherein the optimum stable waveform portion determination unit determines, as the optimum stable waveform portion, a stable waveform portion having a smallest variation among those of the plurality of stable waveform portions.

11. The ultrasound diagnostic device according to claim 1, wherein the period information calculation unit calculates the period information elements from the optimum stable waveform portion.

12. An ultrasound image processing method, comprising:
a step of receiving a frame sequence generated based on a signal obtained by transmitting and receiving an ultrasound wave to and from a periodically moving organ and setting a group of candidate regions for each frame in the frame sequence;
a step of successively calculating, for each of the candidate regions, correlation values between a reference frame and remaining frames other than the reference frame in the frame sequence to generate a correlation value waveform representing a change in the correlation values with respect to time for each of the candidate regions;
a step of localizing a stable waveform portion in the correlation value waveform for each of the candidate regions;
a step of determining an optimum stable waveform portion from among a plurality of the stable waveform portions localized in a plurality of the generated correlation value waveforms, and
a step of calculating, based on the correlation value waveform obtained from one of the candidate regions that corresponds to the optimum stable waveform portion, period information on a motion of the organ.
